(19) United States
(12) Patent Application Publication
(10) Pub. No.: US 2021/0069464 A1
(43) Pub. Date: Mar. 11, 2021

(54) HUBS FOR MEDICAL DEVICES

(71) Applicants: STRYKER CORPORATION, Kalamazoo, MI (US); STRYKER EUROPEAN OPERATIONS LIMITED, Carrigtwohill, (IE)

(72) Inventor: Larry Willie CABALLERO, Courtbrack (IE)

(21) Appl. No.: 16/561,817

(22) Filed: Sep. 5, 2019

(51) Int. Cl. A61M 25/00

(52) U.S. Cl. CPC A61M 25/0014; A61M 25/1025; A61M 25/0097

(57) ABSTRACT

A method of making a medical device includes holding a spacer tool by a slot of a mold, and accommodating a portion of a first tube by a lumen in the spacer tool, wherein a region of the first tube is inside the mold cavity and outside the spacer tool, and an end of the first tube is in engagement with an end of a first lumen-defining section of a first core pin. The spacer tool includes a first section located in the mold cavity when the spacer tool is held by the slot of the mold, the first section of the spacer tool being configured to define a cavity to be formed circumferentially around a part of the first tube inside a hub to be formed, wherein the part of the first tube is different from the region of the first tube.

### FIELD

The field of the application relates to hubs for medical devices, medical devices including such hubs, and methods of making the hubs and the medical devices.

### BACKGROUND

Medical devices, such as stent delivery catheters or other types of catheters, may sometimes include hubs at their proximal ends. In some cases, the hub may be a hub, which includes a first port and a second port. The ports may be used for delivery of substance(s) to a patient, and/or for retrieval of substance(s) from the patient.

Sometimes, a catheter may be made by gluing tubes to a hub. However, using only glue to connect tubes to the hub pose a risk for stent transfer as misalignment within the hub, or variation in the hub/tube dimensions, may restrict or damage a stent when transferring the stent into the catheter tube.

Also, making a catheter with multiple concentric tubes that connect with respective ports of a hub is difficult. This is because the proximal end of an inner catheter tube needs to exit out of the proximal end of an outer catheter tube, while the outer catheter tube maintains fluid communication with its corresponding port at the hub. These features are very difficult to achieve, especially with over-molding techniques.

New manufacturing techniques and tools for making hubs for multi-lumen catheters, and the resulting hubs and catheters made from such new techniques and new tools, are described herein.

### SUMMARY

In accordance with some embodiments, a hub that has a first port and a second port is provided. The first port extends to a first lumen in the hub, and the second port extends to a second lumen in the hub. The first lumen of the hub is in fluid communication with a first tube lumen of a first (inner) tube, and the second lumen of the hub is in fluid communication with a second tube lumen of a second (outer) tube that concentrically surrounds the first tube. The hub is over-molded onto the first tube, and the second tube is attached to the hub after the hub is made. This has the benefit of providing a smooth transition from the first lumen of the hub to the inner surface of the first tube defining the first tube lumen. In some cases, the hub may be a part of a stent delivery catheter. In such cases, the first tube may be configured to deliver stent(s). Because the over-molding technique described herein provides a smooth transition between the first port and the inner surface defining the lumen of the first tube, stent(s) will not be damaged or restricted as it is being transferred into the first tube. The space between the second tube and the first tube may be used to deliver a substance (such as irrigation fluid, inflation fluid for inflating a balloon at a distal end of the stent catheter, drugs, etc.), and/or to retrieve a substance (e.g., irrigation fluid from inside a patient, fluid from a balloon to deflate the balloon, biological samples, etc.). In other cases, the catheter may be other types of catheter.

A medical device, includes: a first tube with a first tube lumen; a second tube with a second tube lumen, wherein the second tube is disposed around the first tube; and a hub comprising a first port, a second port, a first lumen, and a second lumen, the first lumen extending from the first port and being in fluid communication with the first tube lumen of the first tube, the second lumen extending from the second port and being in fluid communication with a cavity within the hub; wherein the hub is made from a material, and wherein the cavity is defined by the material of the hub, and is in fluid communication with a space between the first tube and the second tube.

Optionally, the cavity is disposed around the first tube.

Optionally, a majority of a length of the cavity is located between an end of the second tube and the second lumen.

Optionally, a part of the cavity extends proximally past the second lumen of the hub.

Optionally, the first lumen of the hub and the second lumen of the hub are defined by the material of the hub.

Optionally, an interior surface of the first lumen of the hub has a smooth transition with respect to an inner wall of the first tube that defines the first tube lumen.

Optionally, the first tube has a length that is disposed within the hub.

Optionally, the medical device further includes a sleeve disposed between the first tube and the material of the hub, wherein the hub is coupled to the first tube via the sleeve.

Optionally, the material of the hub defines a space for receiving the second tube.

Optionally, the space has a cross-sectional dimension that is larger than a cross-sectional dimension of the cavity.

Optionally, the hub is formed around a part of the first tube.

Optionally, the second tube is mechanically secured to the hub.

Optionally, the second tube is mechanically secured to the hub via an adhesive.

Optionally, the first tube lumen of the first tube is sized for delivering a stent.

Optionally, the medical device further includes a balloon, wherein the space between the first tube and the second tube is in fluid communication with an interior space of the balloon.

A mold system for making a medical device, includes: a mold having a mold cavity for receiving a material; a first core pin having a first lumen-defining section located, or configured for placement, in the mold cavity; a second core pin having a second lumen-defining section located, or configured for placement, in the mold cavity; and a spacer tool having a lumen sized for accommodating a first tube when the first tube is in a fixed position with respect to the mold, and wherein when the first tube is in the fixed position with respect to the mold, an end of the first tube is engaged with the first lumen-defining section of the first core pin; and wherein the spacer tool comprises a first section configured for placement in the mold cavity of the mold, the first section of the spacer tool having a first outer cross-sectional dimension that is larger than an outer cross-sectional dimension of the first tube.

Optionally, the first section of the spacer tool is in abutment against the second lumen-defining section of the second core pin.

Optionally, an end of the first section of the spacer tool is longitudinally located between an end of the first lumen-defining section of the first core pin and an end of the second lumen-defining section of the second core pin.

Optionally, when the first tube is in the fixed position with respect to the mold, a region of the first tube is outside the spacer tool.

Optionally, the first section of the spacer tool is configured to define a cavity to be formed circumferentially around a part of the first tube inside a hub of the medical device to be formed.

Optionally, the spacer tool further comprises a second section with a second outer cross-sectional dimension that is larger than the first outer cross-sectional dimension of the first section.

Optionally, the second section of the spacer tool is configured to define a space for accommodation of a second tube.

Optionally, a difference between the second outer cross-sectional dimension of the second section of the spacer tool, and the first outer cross-sectional dimension of the first section of the spacer tool, is more than two times a wall thickness of the second tube.

Optionally, the mold comprises a slot, and wherein the spacer tool further comprises a third section configured for placement in the slot of the mold, the slot configured to prevent transverse movement of the spacer tool with respect to the mold.

Optionally, the spacer tool further comprises an end section configured to prevent longitudinal movement of the spacer tool with respect to the mold.

Optionally, the mold comprises a first slot for holding the first core pin, and a second slot for holding the second core pin.

Optionally, the first slot is configured to prevent transverse movement of the first core pin with respect to the mold, and the second slot is configured to prevent transverse movement of the second core pin with respect to the mold.

Optionally, the first core pin comprises a first intermediate section, and the first slot is configured to hold the first intermediate section of the first core pin; and wherein the second core pin comprises a second intermediate section, and the second slot is configured to hold the second intermediate section of the second core pin.

Optionally, the first core pin comprises a first end section configured to prevent longitudinal movement of the first core pin with respect to the mold, and the second core pin comprises a second end section configured to prevent longitudinal movement of the second core pin with respect to the mold.

A method of making a medical device, includes: holding a spacer tool by a slot of a mold, the mold having a mold cavity; and accommodating a portion of a first tube by a lumen in the spacer tool while the first tube is at a fixed position with respect to the mold; wherein when the first tube is at the fixed position with respect to the mold, a region of the first tube is inside the mold cavity and is outside the spacer tool, and an end of the first tube is in engagement with an end of a first lumen-defining section of a first core pin; wherein the spacer tool comprises a first section that is located in the mold cavity when the spacer tool is held by the slot of the mold, the first section of the spacer tool configured to define a cavity to be formed circumferentially around a part of the first tube inside a hub to be formed, wherein the part of the first tube is different from the region of the first tube; and wherein the method further comprises receiving a material by the mold cavity.

Optionally, the method further includes holding the first core pin by the mold, wherein the first core pin is configured to define a first lumen to be formed in the hub.

Optionally, the method further includes preventing the first core pin from longitudinally moving with respect to the mold.

Optionally, the method further includes holding a second core pin by the mold.

Optionally, the method further includes preventing the second core pin from longitudinally moving with respect to the mold.

Optionally, the second core pin is configured to define a second lumen to be formed in the hub.

Optionally, after the hub is made, the second lumen is in fluid communication with the cavity.

Optionally, the method further includes undergoing a curing process by the material to obtain a cured material for the hub.

Optionally, the cured material is in contact with the region of the first tube, and forms the space around the part of the first tube.

Optionally, the region of the first tube comprises a sleeve coupled to a wall of the first tube.

Optionally, the method further includes defining a space, by the spacer tool, to be formed in the hub, wherein the space is sized for accommodating a second tube.

Optionally, the space defined by the spacer tool has a cross-sectional dimension that is larger than a cross-sectional dimension of the cavity.

Optionally, the method further includes receiving the second tube by the space in the hub after the hub is made.

Optionally, the method further includes attaching the second tube to the hub using an adhesive.

Other and further aspects and features will be evident from reading the following detailed description.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 illustrates a transparent view of a mold system for making a hub in accordance with some embodiments, particularly showing the mold system having a mold, two core pins, and a spacer tool.

FIG. 2 illustrates a solid view of the mold of FIG.1.

FIG. 3 illustrates the spacer tool of FIG. 1.

FIG. 4 illustrates an internal part of a hub made using the mold system of FIG. 1.

FIG. 5 illustrates a hub made using the mold system of FIG. 1, particularly showing a position of the spacer tool before it is removed from the hub.

FIG. 6 illustrates a hub made using the mold system of FIG. 1, particularly showing the spacer tool being removed from the hub.

FIG. 7 illustrates a hub made using the mold system of FIG. 1, particularly showing a second tube being attached to the hub after the spacer tool has been removed from the hub.

FIG. 8 illustrates a method of making a medical device using the mold system of FIG. 1.

### DETAILED DESCRIPTION

Various embodiments are described hereinafter with reference to the figures. It should be noted that the figures are not drawn to scale and that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

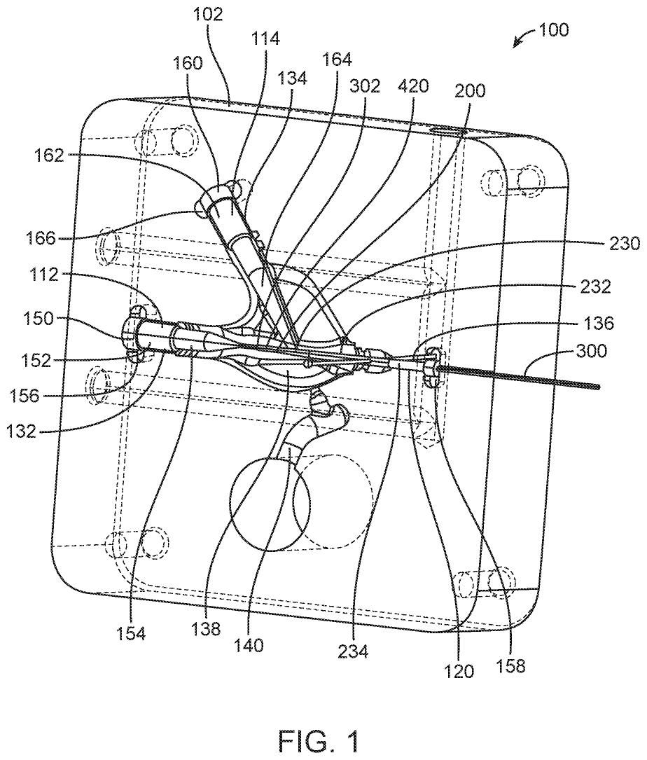

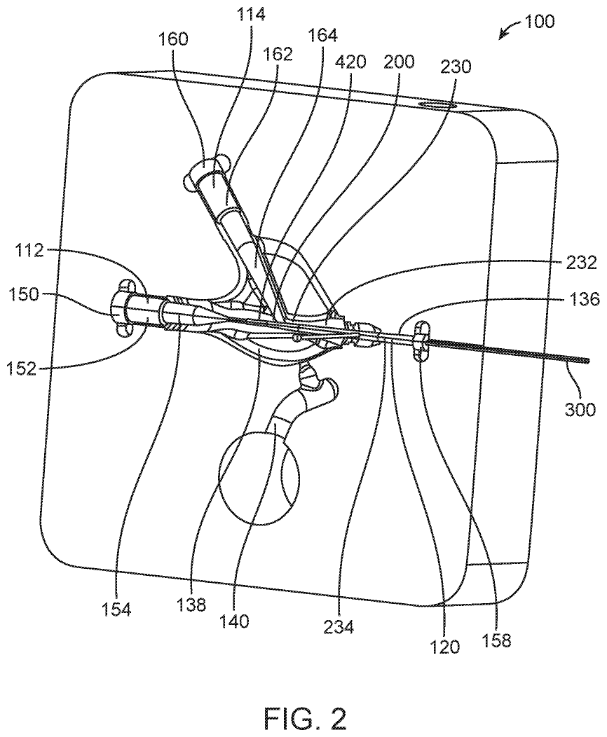

FIGS. 1-2 illustrate respectively a transparent view and a solid view of a mold system 100 for making a hub of a catheter in accordance with some embodiments. The mold system 100 includes a mold 102, a first core pin 112, a second core pin 114, and a spacer tool 120.

The mold 102 includes a first slot 132 configured to accommodate a part of the first core pin 112, a second slot 134 configured to accommodate a part of the second core pin 114, and a third slot 136 configured to accommodate a part of the spacer tool 120. The mold 102 also includes a mold cavity 138 defining a shape of the hub that is to be made from the mold 102. Accordingly, the mold cavity 138 has a shape that corresponds with a shape of the hub that is to be made. As shown in the figure, the mold 102 also includes a delivery channel 140 configured to deliver a material into the mold cavity 138 of the mold 102 to thereby make the hub of the catheter.

The first core pin 112 is configured to occupy a space in the mold cavity 138 that will become a first lumen of a hub to be formed, and the second core pin 114 is configured to occupy a space in the mold cavity 138 that will become a second lumen of the hub to be formed. As used in this specification, the term “core pin” refers to any object that can occupy a space in the mold cavity 138, so that material in the mold cavity 138 can flow around the object to thereby create a desired shape or configuration. The term “core pin” is not limited to an object having any particular shape, and is certainly not limited to an object having a pin configuration. The first core pin 112 includes an end section 150, an intermediate section 152, and a lumen-defining section 154. Similarly, the second core pin 114 includes an end section 160, an intermediate section 162, and a lumen-defining section 164.

The intermediate section 152 of the first core pin 112 is configured (e.g., sized and/or shaped) to fit within the slot 132 of the mold 102 during use. The intermediate section 152 of the first core pin 112 is in abutment against a surface of the first slot 132 so that the first core pin 112 is prevented from moving transversely with respect to the first slot 132. Similarly, the intermediate section 162 of the second core pin 114 is configured (e.g., sized and/or shaped) to fit within the slot 134 of the mold 102 during use. The intermediate section 162 of the second core pin 114 is in abutment against a surface of the second slot 134 so that the second core pin 114 is prevented from moving transversely with respect to the second slot 134.

The end section 150 of the first core pin 112 is configured to prevent the first core pin 112 from moving longitudinally (along an axis of the first core pin 112) with respect to the mold 102. In particular, the cross-sectional dimension of the end section 150 of the core pin 112 is larger than the size of the first slot 132. This allows the end section 150 of the first core pin 112 to abut against an exterior surface of the mold 102 while the end section 150 of the first core pin 112 is accommodated within an opening 156 of the mold 102. Similarly, the end section 160 of the second core pin 114 is configured to prevent the second core pin 114 from moving longitudinally (along an axis of the second core pin 114) with respect to the mold 102. In particular, the cross-sectional dimension of the end section 160 of the second core pin 114 is larger than the size of the second slot 134. This allows the end section 160 of the second core pin 114 to abut against an exterior surface of the mold 102 while the end section 160 of the second core pin 114 is accommodated within an opening 166 of the mold 102.

As shown in FIGS. 1-2, the intermediate section 152 of the first core pin 112, when placed in the first slot 132 of the mold 102, places the lumen-defining section 154 of the first core pin 112 away from interior surfaces of the mold cavity 138. This creates a spacing between the lumen-defining section 154 of the first core pin 112 and the interior surface of the mold cavity 138. Accordingly, when a material is delivered into the mold cavity 138, the material will flow around the lumen-defining section 154 of the first core pin 112 to create a first lumen for the hub. Similarly, the intermediate section 162 of the second core pin 114, when placed in the second slot 134 of the mold 102, places the lumen-defining section 164 of the second core pin 114 away from interior surfaces of the mold cavity 138. This creates a spacing between the lumen-defining section 164 of the second core pin 114 and the interior surface of the mold cavity 138. Accordingly, when a material is delivered into the mold cavity 138, the material will flow around the lumen-defining section 164 of the second core pin 114 to create a second lumen for the hub.

In other embodiments, the first core pin 112 may not include the end section 150, and the second core pin 114 may not include the end section 160. Instead, for example, the intermediate section 152 of the first core pin 112 may have a tapered profile to prevent the first core pin 112 from moving longitudinally with respect to the mold 102. Similarly, in other embodiments, the intermediate section 162 of the second core pin 114 may have a tapered profile to prevent the second core pin 114 from moving longitudinally with respect to the mold 102.

In further embodiments, the first core pin 112 and the second core pin 114 may be made as parts of the mold 102. In such cases, the first core pin 112, the second core pin 114, and the mold 102 have a unity configuration. The lumen-defining section 154 of the first core pin 112 and the lumen-defining section 164 of the second core pin 114 are located in the mold cavity 138 of the mold 102.

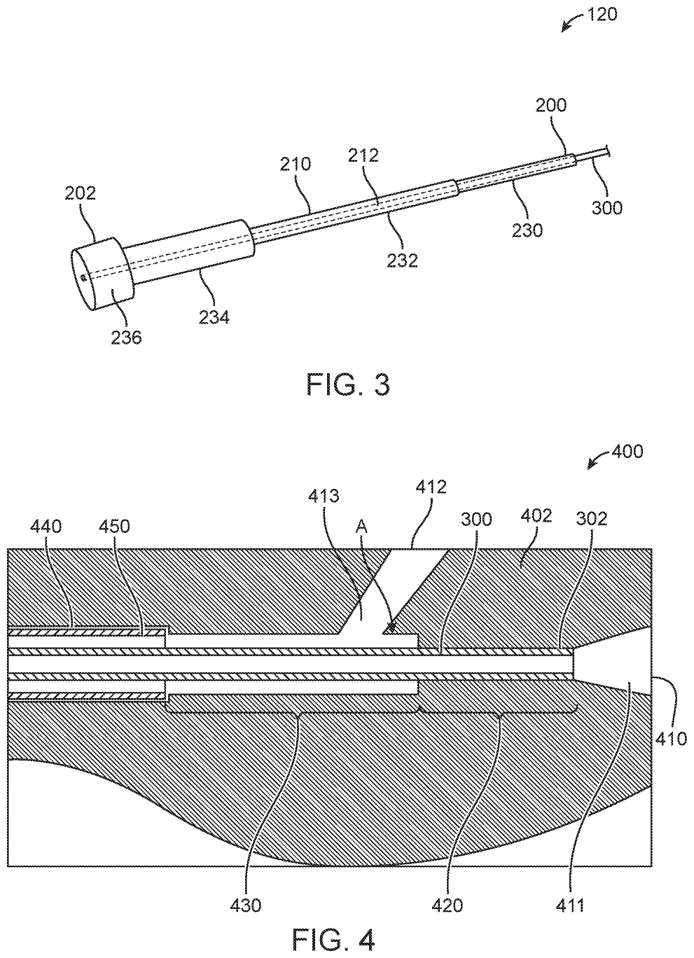

FIG. 3 illustrates the spacer tool 120 in accordance with some embodiments. The spacer tool 120 includes a first end 200, a second end 202, and a body 210 extending between the first end 200 and the second end 202. The spacer tool 120 also includes a lumen 212 in the body 210 extending from the first end 200 to the second end 202. The body 210 of the spacer tool 120 has three sections 230, 232, 234, and an end section 236. The first section 230 has a first outer cross-sectional dimension, the second section 232 has a second outer cross-sectional dimension, and the third section 234 has a third outer cross-sectional dimension. In the illustrated embodiments, the third outer cross-sectional dimension of the third section 234 is larger than the second outer cross-sectional dimension of the second section 232, and the second outer cross-sectional dimension of the second section 232 is larger than the first outer cross-sectional dimension of the first section 230. Also, in the illustrated embodiments, the first section 230, the second section 232, and the third section 234 have respective circular cross-sections. Accordingly, the first outer cross-sectional dimension, the second outer cross-sectional dimension, and the third outer cross-sectional dimension are respective first outer diameter, second outer diameter, and third outer diameter. In other embodiments, the first section 230, the second section 232, or the third section 234 may have other cross-sectional shapes.

As shown in FIGS. 1-4, the lumen 212 of the spacer tool 120 is sized to accommodate a first tube 300 to which the hub is to be over-molded. The lumen 212 has a cross-section that is uniform along the entire length of the body 210 of the spacer tool 120. Also, the first section 230 of the spacer tool 120 has an outer cross-sectional dimension that is larger than the outer cross-sectional dimension of the first tube 300. This configuration allows a cavity (hub cavity) to be created in the hub, wherein the cavity is in fluid communication with the space between outside the inner first tube 300, and also in fluid communication with the second port of the hub to be made. The second section 232 of the spacer tool 120 has an outer cross-sectional dimension that corresponds with an outer cross-sectional dimension of a second tube to be disposed concentrically around the first tube 300 after the hub is overmolded onto the first tube 300. As shown in FIGS. 1-2, the third section 234 of the spacer tool 120 is sized for placement in the third slot 136 of the mold. In particular, the third section 234 of the spacer tool 120 is in abutment against the surface of the third slot 136. This configuration prevents the spacer tool 120 from moving transversely with respect to the mold 102.

The end section 236 of the spacer tool 120 is configured to prevent the spacer tool 120 from moving longitudinally (along a longitudinal axis of the spacer tool 120) with respect to the mold 102. In particular, the cross-sectional dimension of the end section 236 of the spacer tool 120 is larger than the size of the third slot 136. This allows the end section 236 of the spacer tool 120 to abut against an exterior surface of the mold 102 while the end section 236 of the spacer tool 120 is accommodated within an opening 158 (shown in FIGS. 1-2) of the mold 102.

In other embodiments, the spacer tool 120 may not include the end section 236. For example, instead, the third section 234 of the spacer tool 120 may have a tapered profile to prevent the spacer tool 120 from moving longitudinally with respect to the mold 102.

During use of the mold system 100 to make a hub, the first core pin 112 and the second core pin 114 are first placed in their operative positions with respect to the mold 102 like that shown in FIG. 1. Also, the first tube 300 is inserted into the lumen 212 of the spacer tool 120, and extends through the spacer tool 120. The spacer tool 120 with the first tube 300 is placed in an operative position with respect to the mold 102 like that shown in FIG. 1. In the illustrated embodiments, the lumen-defining section 154 of the first core pin 112 has an end that is sized for insertion into an end 302 of the first tube 300. This configuration allows the hub material to flow around the lumen-defining section 154 and up to the edge around the tip opening at the end 302 of the first tube 300, thereby creating a smooth transition with respect to the edge around the tip opening of the first tube 300. Also, as shown in FIGS. 1-3, the first end 200 of the spacer tool 120 does not extend all the way to the first core pin 112, thereby leaving an exposed region 420 of the first tube 300. The exposed region 420 allows hub material delivered into the mold cavity 138 to make contact with the first tube 300, thereby over-molding onto the first tube 300. As can be seen from FIGS. 1-2, the first end 200 of the spacer tool 120 extends past the end of the second core pin 114. This allows a cavity to be created in the hub so that the cavity is in fluid communication with a lumen of a port created by the second core pin 114. This aspect will be further described with reference to FIG. 4.

Next, material used to form the hub is delivered into the mold cavity 138 of the mold 102 via the channel 140 of the mold 120. The hub material will flow around the lumen-defining section 154 of the first core pin 112, around the lumen-defining section 164 of the second core pin 114, around the exposed region 420 of the first tube 300, and around the spacer tool 120. After the hub material is cured, the mold 102, the first and second core pins 112, 114, and the spacer tool 120 are then removed from the created hub, which has been over-molded onto the first tube 300.

Referring now to FIG. 4, which shows the inside of the hub 400 that has been made. As shown in the figure, the hub 400 includes a first port 410 with a first lumen 411 defined by the lumen-defining section 154 of the first core pin 112 (which has been removed from the formed hub 400). The hub 400 also includes a second port 412 with a second lumen 413 defined by the lumen-defining section 164 of the second core pin 114 (which has been removed from the formed hub 400).

Also, as shown in FIG. 4, because the first tube 300 has the exposed region 420 that was not covered by the spacer tool 120 during the molding process, the material 402 of the hub 400 was able to flow around the exposed region 420 and made contact with the exposed region 420 of the first tube 300. This allows the hub 400 to be over-molded onto the exposed region 420 of the first tube, attaching the hub 400 with the exposed region 420 of the first tube 300.

In some embodiments, the exposed region 420 of the first tube 300 may optionally include a sleeve coupled to an external surface of a wall of the first tube 300. The sleeve may protect the wall of the first tube 300 from being damaged by the heat generated during a molding process—e.g., heat from hot material surrounding the first tube 300.

In the illustrated embodiments shown in FIG. 4, the transition between the lumen 411 and the inner surface of the first tube 300 has a smooth transition. This allows a substance, such as a stent, to be delivered into the first tube 300 from the lumen 411 without blockage or restriction. In some cases, the first tube 300 may be used to deliver a stent distally into a patient. In other cases, the first tube 300 may be used to transport a stent (e.g., a stent removed from a patient) proximally into the lumen 411.

FIG. 4 also shows a cavity 430 formed within the hub 400. The cavity 430 surrounds a part of the first tube 300, and is in fluid communication with the lumen 413 of the second port 412. The cavity 430 is defined by the hub material 402, which flowed around the first section 230 of the spacer tool 120 during the molding process. After the hub 400 is formed, the spacer tool 120 was removed, thereby leaving the cavity 430 around a part of the first tube 300, with the wall of the cavity spaced away from the first tube 300. The longitudinal length of the cavity 430 in the hub 400 corresponds with (e.g., is equal to) the length of the first section 230 of the spacer tool 120.

As discussed, during the molding process, the first end 200 of the spacer tool 120 extends past the end of the second core pin 114. This allows a cavity to be created in the hub so that the cavity is in fluid communication with a lumen of a port created by the second core pin 114. This aspect can be seen in FIG. 4, which shows the cavity 430 in the hub 400, with the cavity 430 extending slightly past the lumen 413 (defined by the second core pin 114 during the molding process). Because the first section 230 of the spacer tool 120 (defining the cavity 430) stops at location A, which past the end of the second core pin 114, the resulting cavity 430 and the resulting lumen 413 will be in fluid communication with each other.

As shown in FIG. 4, the formed hub 400 also includes a space 440 that functions as a tube-receiving section for receiving a second tube 450. The space 440 is larger than the cavity 430 because the space 440 is defined by the second section 232 of the spacer tool 120, which has a larger outer cross-sectional dimension than that of the first section 230 (which defines the cavity 430). In particular, during the molding process, the hub material 402 flows around the second segment 232 of the spacer tool 120, which occupies the area that will become the space 440. The hub material 402 also flows around the first segment 230 of the spacer tool 120, which occupies the space that will become the cavity 430. Because the second section 232 of the spacer tool 120 is larger than the first section 230, the space 440 for receiving the second tube 450 is also larger than the cavity 430. As shown in the figure, after the hub 400 is formed, the second tube 450 is then inserted into the space 440. The second tube 450 may be mechanically attached to the hub 400 using an adhesive (such as glue, epoxy, etc.) or a mechanical connector. The second tube 450 is concentrically disposed around the first tube 300, with an inner surface of the second tube 450 and the outer surface of the first tube 300 defining a space.

When using the hub 400 to deliver a substance distally via the second port 412, the substance is delivered through the second port 412 of the hub 400 and travels through the lumen 413. The substance is delivered into the cavity 430 defined by the hub material 402. The substance then travels from within the cavity 430 into the space 440 between the first and second tubes 300, 450.

When using the hub 400 to deliver a substance proximally via the second port 412, the substance travels proximally through the space 440 between the first and second tubes 300, 450. The substance then enters into the cavity 430 defined by the hub material 402, and travels into the lumen 413. The substance then exits from the second port 412.

In the illustrated embodiments, the inner cross-sectional dimension of the second tube 450 is larger than the outer cross-sectional dimension of the first tube 300 to thereby create the space between the first tube 300 and the second tube 450. In some cases, the inner cross-sectional dimension of the second tube 450 may be equal to the cross-sectional dimension of the cavity 430. Such may be accomplished by making the inner cross-sectional dimension of the second tube 450, and the outer cross-sectional dimension of the first section 230 (for defining the cavity 430) of the spacer tool 120, equal to each other. In other cases, the inner cross-sectional dimension of the second tube 450 may be larger than the cross-sectional dimension of the cavity 430. This has the benefit of ensuring that substance (traveling in the distal direction) delivered from the second port 412 into the cavity 430 will travel into the space between the first and second tube 300, 450 without blockage or restriction. In further cases, the inner cross-sectional dimension of the second tube 450 may be smaller than the cross-sectional dimension of the cavity 430. This has the benefit of ensuring that substance (traveling in the proximal direction) delivered from within the space between the first and second tubes 300, 450 can travel into the cavity 430 without blockage or restriction.

The space 440 between the first and second tubes 300, 450 may be used to deliver a substance (e.g., irrigation fluid, drug, etc.) from the port 412 to a patient. Additionally or alternatively, the space 440 between the first and second tubes 300, 450 may be used to remove a substance (e.g., irrigation fluid, biological samples, etc.) from the patient. Also, in some embodiments, the space 440 between the first and second tubes 300, 450 may be used to deliver inflation fluid to inflate a balloon at a distal end of a medical device (that includes the hub 400), and/or to remove inflation fluid from a balloon to deflate the balloon.

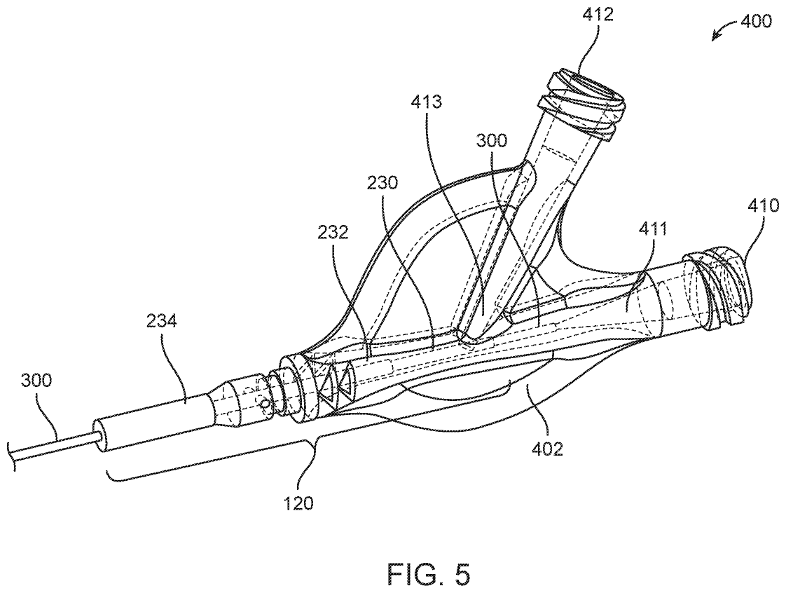

FIG. 5 shows the hub 400 that has been formed, with the mold 102 and core pins 112, 114 removed. However, the spacer tool 120 has not yet been removed. The respective positions of the first section 230, the second section 232, and the third section 234 of the spacer tool 120 can be seen in relation to the hub 400. The end section 236 of the spacer tool 120 is not shown for clarity purpose. The formed first and second lumens 411, 413 inside the hub 400 are also shown. The first lumen 411 in the hub 400 is in fluid communication with the first tube 300. The second lumen 413 ends at an exterior surface of the first section 230 of the spacer tool 120.

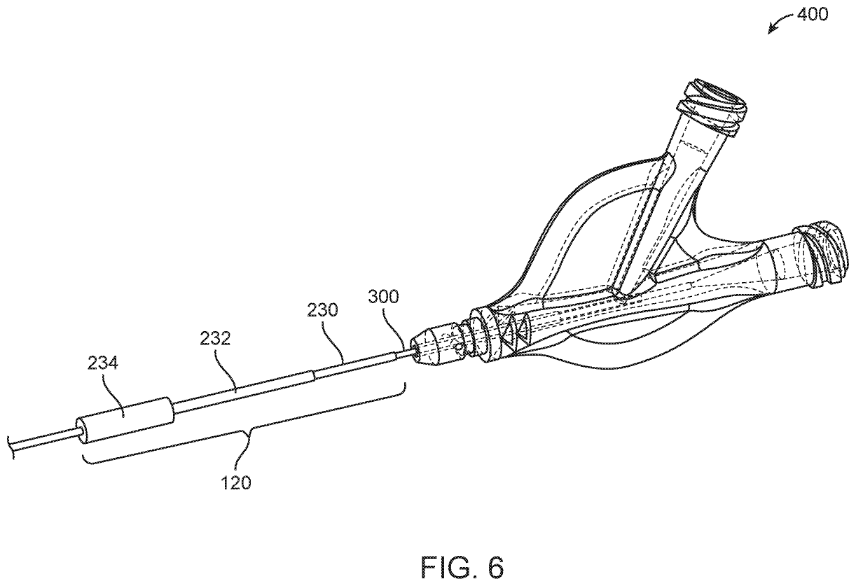

FIG. 6 shows the hub 400 of FIG. 5, particularly showing the spacer tool 120 being removed.

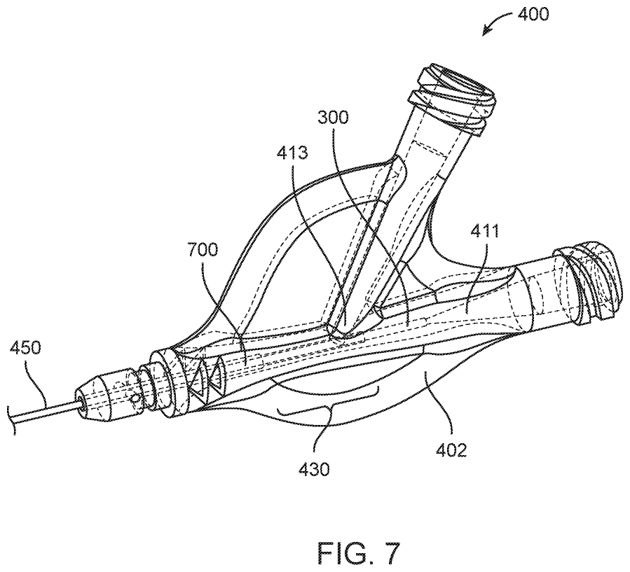

FIG. 7 shows the hub 400 of FIG. 5, particularly showing the second tube 450 being attached to the hub 400 after the spacer tool 120 has been completely removed. The cavity (hub cavity) 430 previously occupied by the first section 230 of the spacer tool 120 is also shown. The cavity 430 is in fluid communication with the second lumen 413 in the hub 400, and is defined by the material 402 of the hub 400. The cavity 430 is also in fluid communication with the space 440 of FIG. 4, that is defined between the outer surface of the first tube 300 and the inner surface of the second tube 450. As shown in the figure, the end 700 of the second tube 450 cannot extend into the cavity 430. This is because the cavity 430 formed by the first section 230 of the spacer tool 120 has a smaller cross-sectional dimension than the space (which now received the second tube 450) formed by the second section 232 of the spacer tool 120.

The hub-forming technique described herein may be used to form any hub for any catheter. For example, the above described technique may be used to form a Y-hub that includes two proximal ports at the hub in communication with two respective tube lumens of catheter tubes. In other embodiments, the above describe technique may be used to form a hub having more than two ports and two hub lumens that are in respective fluid communication with more than two catheter tube lumens. For example, in other embodiments, the spacer tool 120 may include additional section(s) configured to define tube-receiving space(s) for additional tube(s) that surrounds the inner first tube 300. In such cases, the mold system 100 may include corresponding additional core pin(s) for defining respective hub lumen(s) that will be in fluid communication with the tube-receiving space(s).

As illustrated in the above embodiments, the mold system 100 and the technique of forming a hub using the mold system 100 are advantageous because they allow the hub to be easily formed while providing the hub with two ports and two respective hub lumens that are in fluid communication with respective tubes (catheter tubes). The resulting hub is also advantageous because the hub has a smooth transition between an inner wall of a tube and an inner surface of a hub lumen.

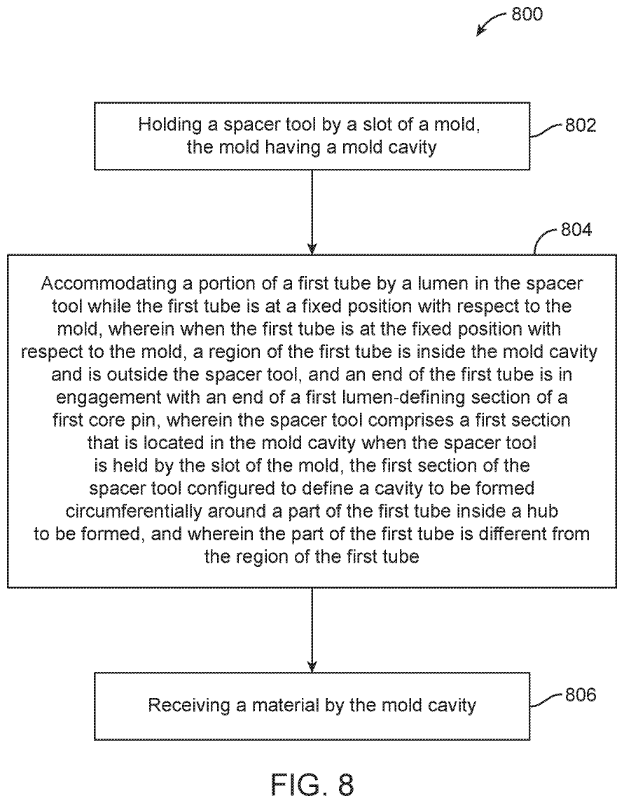

FIG. 8 illustrates a method 800 of making a medical device. As used in this specification, the term “medical device” may refer to one or more component(s) of a device for medical use, or a finished manufactured device itself. For example, the medical device, may be a hub, a component of a catheter having a hub and one or more tubes attached thereto, or the catheter itself. The method 800 includes holding a spacer tool by a slot of a mold, the mold having a mold cavity (item 802). The method 800 also includes accommodating a portion of a first tube by a lumen in the spacer tool while the first tube is at a fixed position with respect to the mold, wherein when the first tube is at the fixed position with respect to the mold, a region of the first tube is inside the mold cavity and is outside the spacer tool, and an end of the first tube is in engagement with an end of a first lumen-defining section of a first core pin, wherein the spacer tool comprises a first section that is located in the mold cavity when the spacer tool is held by the slot of the mold, the first section of the spacer tool configured to define a cavity to be formed circumferentially around a part of the first tube inside a hub to be formed, and wherein the part of the first tube is different from the region of the first tube (item 804). The method 800 further includes receiving a material by the mold cavity (item 806).

Optionally, the method 800 further includes holding the first core pin by the mold, wherein the first core pin is configured to define a first lumen to be formed in the hub.

Optionally, the method 800 further includes preventing the first core pin from longitudinally moving with respect to the mold.

Optionally, the method 800 further includes holding a second core pin by the mold.

Optionally, the method 800 further includes preventing the second core pin from longitudinally moving with respect to the mold.

Optionally, in the method 800 the second core pin is configured to define a second lumen to be formed in the hub.

Optionally, in the method 800, after the hub is made, the second lumen is in fluid communication with the cavity.

Optionally, the method 800 further includes undergoing a curing process by the material to obtain a cured material for the hub.

Optionally, in the method 800, the cured material is in contact with the region of the first tube, and forms the space around the part of the first tube.

Optionally, in the method 800, the region of the first tube comprises a sleeve coupled to a wall of the first tube.

Optionally, the method 800 further includes defining a space, by the spacer tool, to be formed in the hub, wherein the space is sized for accommodating a second tube.

Optionally, in the method 800, the space defined by the spacer tool has a cross-sectional dimension that is larger than a cross-sectional dimension of the cavity.

Optionally, the method 800 further includes receiving the second tube by the space in the hub after the hub is made.

Optionally, the method 800 further includes attaching the second tube to the hub using an adhesive.

The following items are exemplary features of embodiments described herein. Each item may be an embodiment itself or may be a part of an embodiment. One or more items described below may be combined with other item(s) in an embodiment.

Item 1: A medical device, includes: a first tube with a first tube lumen; a second tube with a second tube lumen, wherein the second tube is disposed around the first tube; and a hub comprising a first port, a second port, a first lumen, and a second lumen, the first lumen extending from the first port and being in fluid communication with the first tube lumen of the first tube, the second lumen extending from the second port and being in fluid communication with a cavity within the hub; wherein the hub is made from a material, and wherein the cavity is defined by the material of the hub, and is in fluid communication with a space between the first tube and the second tube.

Item 2: In the medical device, the cavity (hub cavity) is disposed around the first tube.

Item 3: In the medical device, a majority of a length of the cavity is located between an end of the second tube and the second lumen (that is in fluid communication with the second port of the hub).

Item 4: In the medical device, a part of the cavity (hub cavity) extends proximally past the second lumen of the hub.

Item 5: In the medical device, the first lumen of the hub and the second lumen of the hub are defined by the material of the hub.

Item 6: In the medical device, an interior surface of the first lumen of the hub has a smooth transition with respect to an inner wall of the first tube that defines the first tube lumen.

Item 7: In the medical device, the first tube has a length that is disposed within the hub.

Item 8: The medical device further includes a sleeve disposed between the first tube and the material of the hub, wherein the hub is coupled to the first tube via the sleeve.

Item 9: In the medical device, the material of the hub defines a space for receiving the second tube.

Item 10: In the medical device, the space has a cross-sectional dimension that is larger than a cross-sectional dimension of the cavity (hub cavity).

Item 11: In the medical device, the hub is formed around a part of the first tube.

Item 12: In the medical device, the second tube is mechanically secured to the hub.

Item 13: In the medical device, the second tube is mechanically secured to the hub via an adhesive.

Item 14: In the medical device, the first tube lumen of the first tube is sized for delivering a stent.

Item 15: The medical device further includes a balloon, wherein the space between the first tube and the second tube is in fluid communication with an interior space of the balloon.

Item 16: A mold system for making a medical device, includes: a mold having a mold cavity for receiving a material; a first core pin having a first lumen-defining section located, or configured for placement, in the mold cavity; a second core pin having a second lumen-defining section located, or configured for placement, in the mold cavity; and a spacer tool having a lumen sized for accommodating a first tube when the first tube is in a fixed position with respect to the mold, and wherein when the first tube is in the fixed position with respect to the mold, an end of the first tube is engaged with the first lumen-defining section of the first core pin; and wherein the spacer tool comprises a first section configured for placement in the mold cavity of the mold, the first section of the spacer tool having a first outer cross-sectional dimension that is larger than an outer cross-sectional dimension of the first tube.

Item 17: In the mold system, the first section of the spacer tool is in abutment against the second lumen-defining section of the second core pin.

Item 18: In the mold system, an end of the first section of the spacer tool is longitudinally located between an end of the first lumen-defining section of the first core pin and an end of the second lumen-defining section of the second core pin.

Item 19: In the mold system, when the first tube is in the fixed position with respect to the mold, a region of the first tube is outside the spacer tool.

Item 20: In the mold system, the first section of the spacer tool is configured to define a cavity to be formed circumferentially around a part of the first tube inside a hub of the medical device to be formed.

Item 21: In the mold system, the spacer tool further comprises a second section with a second outer cross-sectional dimension that is larger than the first outer cross-sectional dimension of the first section.

Item 22: In the mold system, the second section of the spacer tool is configured to define a space for accommodation of a second tube.

Item 23: In the mold system, a difference between the second outer cross-sectional dimension of the second section of the spacer tool, and the first outer cross-sectional dimension of the first section of the spacer tool, is more than two times a wall thickness of the second tube.

Item 24: In the mold system, the mold comprises a slot, and wherein the spacer tool further comprises a third section configured for placement in the slot of the mold, the slot configured to prevent transverse movement of the spacer tool with respect to the mold.

Item 25: In the mold system, the spacer tool further comprises an end section configured to prevent longitudinal movement of the spacer tool with respect to the mold.

Item 26: In the mold system, the mold comprises a first slot for holding the first core pin, and a second slot for holding the second core pin.

Item 27: In the mold system, the first slot is configured to prevent transverse movement of the first core pin with respect to the mold, and the second slot is configured to prevent transverse movement of the second core pin with respect to the mold.

Item 28: In the mold system, the first core pin comprises a first intermediate section, and the first slot is configured to hold the first intermediate section of the first core pin; and wherein the second core pin comprises a second intermediate section, and the second slot is configured to hold the second intermediate section of the second core pin.

Item 29: In the mold system, the first core pin comprises a first end section configured to prevent longitudinal movement of the first core pin with respect to the mold, and the second core pin comprises a second end section configured to prevent longitudinal movement of the second core pin with respect to the mold.

Item 30: A method of making a medical device, includes: holding a spacer tool by a slot of a mold, the mold having a mold cavity; and accommodating a portion of a first tube by a lumen in the spacer tool while the first tube is at a fixed position with respect to the mold; wherein when the first tube is at the fixed position with respect to the mold, a region of the first tube is inside the mold cavity and is outside the spacer tool, and an end of the first tube is in engagement with an end of a first lumen-defining section of a first core pin; wherein the spacer tool comprises a first section that is located in the mold cavity when the spacer tool is held by the slot of the mold, the first section of the spacer tool configured to define a cavity to be formed circumferentially around a part of the first tube inside a hub to be formed, wherein the part of the first tube is different from the region of the first tube; and wherein the method further comprises receiving a material by the mold cavity.

Item 31: The method further includes holding the first core pin by the mold, wherein the first core pin is configured to define a first lumen to be formed in the hub.

Item 32: The method further includes preventing the first core pin from longitudinally moving with respect to the mold.

Item 33: The method further includes holding a second core pin by the mold.

Item 34: The method further includes preventing the second core pin from longitudinally moving with respect to the mold.

Item 35: In the method, the second core pin is configured to define a second lumen to be formed in the hub.

Item 36: In the method, after the hub is made, the second lumen is in fluid communication with the cavity.

Item 37: The method further includes undergoing a curing process by the material to obtain a cured material for the hub.

Item 38: In the method, the cured material is in contact with the region of the first tube, and forms the space around the part of the first tube.

Item 39: In the method, the region of the first tube comprises a sleeve coupled to a wall of the first tube.

Item 40: The method further includes defining a space, by the spacer tool, to be formed in the hub, wherein the space is sized for accommodating a second tube.

Item 41: In the method, the space defined by the spacer tool has a cross-sectional dimension that is larger than a cross-sectional dimension of the cavity.

Item 42: The method further includes receiving the second tube by the space in the hub after the hub is made.

Item 43: The method further includes attaching the second tube to the hub using an adhesive.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents.

1. A method of making a medical device, comprising:
holding a spacer tool by a slot of a mold, the mold having a mold cavity;
accommodating a portion of a first tube by a lumen in the spacer tool while the first tube is at a fixed position with respect to the mold, wherein when the first tube is at the fixed position with respect to the mold, a region of the first tube is inside the mold cavity and is outside the spacer tool, and an end of the first tube is in engagement with an end of a first lumen-defining section of a first core pin, the spacer tool comprising a first section that is located in the mold cavity when the spacer tool is held by the slot of the mold, the first section of the spacer tool configured to define a cavity to be formed circumferentially around a part of the first tube inside a hub to be formed, wherein the part of the first tube is different from the region of the first tube; and
receiving a material by the mold cavity.

2. The method of claim 1, further comprising holding the first core pin by the mold, wherein the first core pin is configured to define a first lumen to be formed in the hub.

3. The method of claim 2, further comprising holding a second core pin by the mold.

4. The method of claim 3, further comprising preventing the second core pin from longitudinally moving with respect to the mold.

5. The method of claim 3, wherein the second core pin is configured to define a second lumen to be formed in the hub.

6. The method of claim 5, wherein after the hub is made, the second lumen is in fluid communication with the cavity.

7. The method of claim 1, further comprising preventing the first core pin from longitudinally moving with respect to the mold.

8. The method of claim 1, further comprising undergoing a curing process by the material to obtain a cured material for the hub.

9. The method of claim 8, wherein the cured material is in contact with the region of the first tube, and forms the space around the part of the first tube.

10. The method of claim 9, wherein the region of the first tube comprises a sleeve coupled to a wall of the first tube.

11. The method of claim 1, further comprising defining a space, by the spacer tool, to be formed in the hub, wherein the space is sized for accommodating a second tube.

12. The method of claim 11, wherein the space defined by the spacer tool has a cross-sectional dimension that is larger than a cross-sectional dimension of the cavity.

13. The method of claim 11, further comprising receiving the second tube by the space in the hub after the hub is made.

14. The method of claim 11, further comprising attaching the second tube to the hub using an adhesive.

15. A method of making a medical device, comprising:
holding a spacer tool by a slot of a mold, the mold having a mold cavity;
accommodating a portion of a first tube by a lumen in the spacer tool while the first tube is at a fixed position with respect to the mold, wherein when the first tube is at the fixed position with respect to the mold, a region of the first tube is inside the mold cavity and is outside the spacer tool, and an end of the first tube is in engagement with an end of a first lumen-defining section of a first core pin, the spacer tool comprising a first section that is located in the mold cavity when the spacer tool is held by the slot of the mold, the first section of the spacer tool configured to define a cavity to be formed circumferentially around a part of the first tube inside a hub to be formed, wherein the part of the first tube is different from the region of the first tube;
receiving a material by the mold cavity; and
defining a space, by the spacer tool, to be formed in the hub, wherein the space is sized for accommodating a second tube and has a cross-sectional dimension that is larger than a cross-sectional dimension of the cavity.

16. The method of claim 15, further comprising receiving the second tube by the space in the hub after the hub is made, and attaching the second tube to the hub using an adhesive.

17. The method of claim 15, further comprising
holding the first core pin by the mold, wherein the first core pin is configured to define a first lumen to be formed in the hub, and
holding a second core pin by the mold
preventing the second core pin from longitudinally moving with respect to the mold.

18. The method of claim 17, wherein the second core pin is configured to define a second lumen to be formed in the hub, and wherein after the hub is made, the second lumen is in fluid communication with the cavity.

19. The method of claim 15, further comprising undergoing a curing process by the material to obtain a cured material for the hub, wherein the cured material is in contact with the region of the first tube, and forms the space around the part of the first tube.
